(19) United States
(12) Patent Application Publication
(10) Pub. No.: US 2021/0220171 A1
(43) Pub. Date: Jul. 22, 2021

(54) Microbead And Nanofiber Based Controlled Drug Release System

(71) Applicant: University of Florida Research Foundation, Inc., Gainesville, FL (US)

(72) Inventors: Yong-Kyu Yoon, Gainesville, FL (US); Sheng-Po Fang, Beaverton, OR (US)

(21) Appl. No.: 17/150,035

(22) Filed: Jan. 15, 2021

Related U.S. Application Data
(60) Provisional application No. 62/962,045, filed on Jan. 16, 2020.

(51) Int. Cl. A61F 9/00

(52) U.S. Cl. CPC A61F 9/0017; D01D 5/0007

(57) ABSTRACT

Various examples are provided for controlled drug release over an extended period of time. In one example, a controlled release system includes a multilayer membrane including a first biocompatible nanofiber layer; a microbead layer disposed on the first biocompatible nanofiber layer, the microbeads comprising a releasable agent; and a second biocompatible nanofiber layer disposed over the microbead layer. The first and second biocompatible nanofiber layers support the microbead layer and provide a diffusion barrier that can control a release profile of the releasable agent.

### CROSS REFERENCE TO RELATED APPLICATIONS

This application claims priority to, and the benefit of, co-pending U.S. provisional application entitled “Microbead and Nanofiber Based Controlled Drug Release System” having Ser. No. 62/962,045, filed Jan. 16, 2020, which is hereby incorporated by reference in its entirety.

### BACKGROUND

Many treatment regimens include the administration of a drug multiple times a day. The high frequency of treatment helps maintain the drug concentrations at effective levels. However, continuing this treatment over extended periods of weeks or months can be a challenge. Micro/nanoscale functional materials in various morphologies ranging from films, spheres, to fibers have gained large attention in many fields such as medicine, biology, electronics, optics, and energy. Recent advances in nanotechnology provide opportunities to develop an optimal drug delivery system. Many ophthalmic diseases can require frequent regular treatments such as eye drops multiple times per day, and the treatments should be repeated over a few weeks to months or even lifetime. This high frequency of instillation is needed due to the short ocular residence time of the drops and often leads to poor compliance. Therefore, controlled release devices which have extended release profiles are greatly desired.

### SUMMARY

Aspects of the present disclosure are related to controlled drug release over an extended period of time. In one aspect, among others, a controlled release system comprises a multilayer membrane comprising: a first biocompatible nanofiber layer; a microbead layer disposed on the first biocompatible nanofiber layer, the microbeads comprising a releasable agent; and a second biocompatible nanofiber layer disposed over the microbead layer. The first and second biocompatible nanofiber layers can support the microbead layer and provide a diffusion barrier configured to control a release profile of the releasable agent. In one or more aspects, the releasable agent can be a drug. The first and second biocompatible nanofiber layers can comprise electrospun nanofibers. The first biocompatible nanofiber layer can comprise electrospun nanofibers having a first diameter and the second biocompatible nanofiber layer can comprise electrospun nanofibers having a second diameter. The first and second biocompatible nanofiber layers can comprise a biodegradable polymer. The biodegradable polymer can be polycaprolactone (PCL).

In various aspects, the microbead layer can comprise microbeads in a volume percentage in a range from about 10% to about 99%. The volume percentage can be in a range from about 25% to about 95%. The volume percentage can be in a range from about 40% to about 91%. The release profile of the releasable agent can comprise an initial burst and a linear release after the initial burst. The release rate of the initial burst can be about 30% or less. The linear release can extend over a period of about 80 days or more. In some aspects, the system can comprise a second microbead layer disposed on the first biocompatible nanofiber layer; and a third biocompatible nanofiber layer disposed over the second microbead layer. The second microbead layer can comprise a second releasable agent. The microbead layer can comprise a second releasable agent.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1E illustrate examples of single and dual syringe electrospinning, in accordance with various embodiments of the present disclosure.

FIG. 2A shows SEM images of electrospun nanofiber membranes with an electric field of 1 kV/cm, a TCD of 10 cm, and various PCL concentrations, in accordance with various embodiments of the present disclosure.

FIG. 2B illustrates an example of Microbead and nanofiber diameter measurement of PCL with varying polymer concentration, in accordance with various embodiments of the present disclosure.

FIG. 3A shows SEM images of fabricated single-phase nanofibers and microbeads membranes, in accordance with various embodiments of the present disclosure.

FIG. 3B illustrates an example of release profiles of the single-phase nanofibers and microbeads membranes of FIG. 3A, in accordance with various embodiments of the present disclosure.

FIG. 4 illustrates a comparison of analytical model prediction and experimental results of microbead diameter with solution concentration of 4%, an electric field of 1 kV/cm, and varying TCD from 7.5 cm to 17.5 cm, in accordance with various embodiments of the present disclosure.

FIGS. 5A and 5B show SEM images of fabricated two-phase hybrid membranes with different morphologies, in accordance with various embodiments of the present disclosure.

FIG. 5C illustrates an example of release profiles of two-phase hybrid structures of FIGS. 5A and 5B, in accordance with various embodiments of the present disclosure.

FIGS. 6A-6C illustrate an example of a multilayer structure comprising nanofibers without drug loading and microbeads with drug loading and a release profile, in accordance with various embodiments of the present disclosure.

### DETAILED DESCRIPTION

Disclosed herein are various examples related to controlled drug release over an extended period of time (e.g., over months to a year or more). Methods, systems and apparatus related to multilayer microbead/nanofiber membranes that can provide controlled drug release over time are presented. A multi-syringe electrospinning system (e.g., comprising dual syringes) can be used to fabricate the multilayer microbead/nanofiber membranes. The construction of the multilayer microbead/nanofiber membrane can control burst and release patterns of the drug during use. The use of the multilayer microbead/nanofiber membranes can simplify and benefit patients and physicians in maintaining a consistent drug level of extended periods of time.

Fabrication of a multiple-layered membrane is disclosed. The multilayer membrane can comprise a dexamethasone (DX)-loaded polycaprolactone (PCL) microbead layer sandwiched between two PCL nanofiber layers, which is demonstrated for highly extended linear drug release with low initial burst. Dual syringe sequential electrospinning can be used to produce the multiple layers. A linear drug release of over 80 days and a significantly reduced initial burst of less than 30% is demonstrated. With the dual syringe electrospinning system, more degrees of freedom in the fabrication of a membrane with a customizable release profile can be realized.

Controlled release devices with extended release time are highly desired. Efforts to address the controlled release issues can include modifications of carrier device structures of contact lens and using different nano/micro morphologies of biodegradable microspheres, hydrogels, and electrospun nanofibers for different ocular disease treatments. However, realization of a prolonged drug release greater than a few weeks with a linear release profile and sufficient drug concentration to penetrate through the cornea for posterior ocular diseases remains a challenge. The disclosed extended controlled release devices are also useful for treatment of inner ear diseases and posterior segment ocular diseases.

The electrospinning technique shows good potential for controlled drug release because its capability of producing micro/nanoscale diameter fibers with high mechanical flexibility, which is critical to minimize possible discomfort when the devices are placed in the eye. Additionally, the morphology of electrospun nanofiber membranes ranges from microscale spheres to nanoscale fibers with variation of solution properties and other electrospinning process parameters. Pure microbead, pure nanofiber, or microbead/nanofiber hybrid membranes can be obtained when the solution is in dilute, semi-dilute entangled, or semi-dilute unentangled conditions, respectively. The hybrid membranes are often considered as structural defects. However, a hybrid structure comprising microbeads and nanofibers has an advantage coming from the different dimensions of the nanofibers and the microbeads. For example, it can lead to different release times of embedded drugs. Moreover, the drug release profile can be controlled by several parameters such as the morphology, porosity, hydrophobicity, crystallinity, and device architecture.

A nanofibers/microbeads hybrid drug release system, where single syringe electrospinning was used, has shown a release profile of 18 days, which is 40 times higher than that of drug embedded nanofibers only. However, the still high initial burst of 60% and a rather parabolic release profile remained an impediment to its clinical usage. In this disclosure, implementation of a triple-layered membrane comprising two nanofiber layers and a microbead layer sandwiched between the two nanofiber layers, all made of polycaprolactone (PCL), a biodegradable polymer, is presented. The nanofiber layers do not contain drug but serve as a mechanical support for the microbeads and a diffusion barrier to drug release. Meanwhile, the embedded microbeads contain the drug to be delivered. A test release profile of over 80 days is demonstrated with a projected release of over 120 days with only 30% initial burst. By changing the thickness of the electrospun nanofiber layers, the burst and release pattern can be further controlled.

Two fabrication methods for microbead/nanofiber composite membranes, such as single syringe electrospinning (SSE) and dual syringe electrospinning (DSE), are reported. Also, membrane morphology effects on the release profile are studied. SSE produces (1) single-phase membranes which are made of either microbeads or nanofibers and (2) two-phase hybrid membranes comprising both microbeads and nanofibers. DSE is exploited for multilayer sandwiched membranes where drug is loaded in a microbead layer, sandwiched between nanofiber barrier layers without drug loading. The effects of polymer concentration and electrospinning parameters on the membrane morphology are examined by scanning electron microscope. Drug release measurements were carried out by soaking the membrane in a phosphate buffered saline (PBS) solution at room temperature under a sink condition.

PCL can be used as a carrier polymer to achieve an extended release of its cargo due to its favorable degradation profile. Dexamethasone (DX), an ocular drug for infection and allergies, was loaded for a controlled release study. Polycaprolactone (PCL, average Mw=80,000), dichloromethane (DCM, ACS reagent, ≥99.5%), dimethylformamide (DMF, anhydrous, 99.8%), and dexamethasone (DX, 98%), were purchased from Sigma Aldrich Chemicals (St. Louis, Mo., USA). PCL was chosen as the carrier polymer to achieve a long-term release profile as it shows a long biodegradation profile. Also, PCL's hydrophobicity leads to a long, sustained release. DX, an ocular drug for infection and allergies, was loaded for a controlled release study. PCL was dissolved at various concentrations in a 1:1 mixture of DCM and DMF to create electrospinning precursors of differing viscosities. DX was then added to the PCL solution at 10% of polymer loading. The DX loaded PCL solutions undergo constant magnetic stirring at 60 rpm overnight at room temperature to obtain a homogeneous condition.

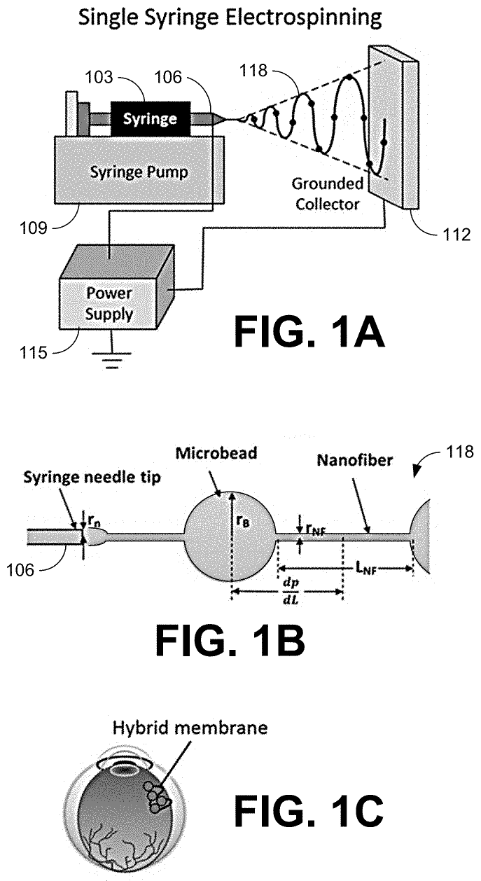

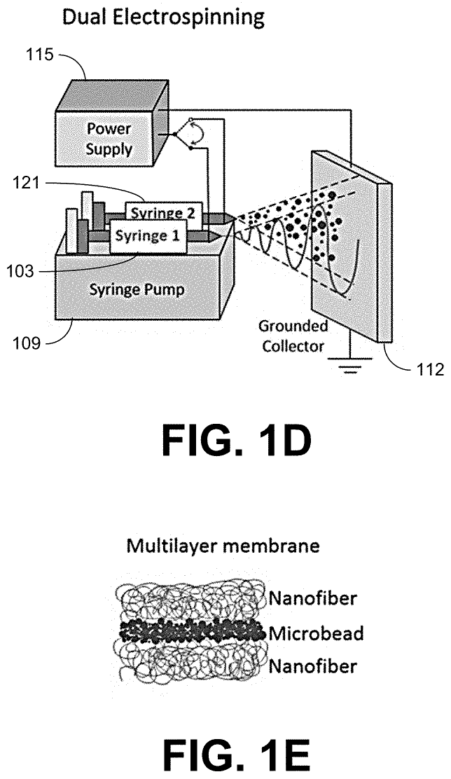

Single-syringe electrospinning. Single syringe electrospinning (SSE) can be used to produce electrospun nanofibers and/or microbeads membranes, where experimental parameters include polymer solution viscosity, solvent choice, electric field strength, and tip-to-collector distance (TCD). FIG. 1A illustrates an example of a single syringe electrospinning (SSE) setup for single-phase and two-phase hybrid membranes. The SSE setup comprises a syringe 103 fitted with, e.g., a 22-gauge needle 106, a syringe pump 109 to provide a constant flow rate of, e.g., 1 ml/hr, a metallic collector plate 112, and a high voltage power supply 115. The needle tip 106 is connected to the positive terminal of the high voltage supply 115, and the metallic collector plate 112 is connected to the ground terminal. A Taylor cone can be formed when the applied voltage exceeds the solution's critical voltage, which is determined by solution parameters. A solution jet 118 can then be ejected toward the collector plate 112. The ejected polymer jet 118 undergoes a series of bending, whipping, splitting, and stretching processes during which the polymer diameter is reduced, and solvent is evaporated. As a result, a charged solid nanoporous polymer mat can be randomly collected on the grounded metallic collector plate 112. The electrospun polymer morphology depends on solution and operating parameters such as the viscosity, applied electric field strength and tip-to-collector distance (TCD).

FIG. 1B illustrates an example of the ejected polymer jet 118. The electrospun membrane morphology can change from microbeads to nanofibers with increasing solution viscosity. In this disclosure, the release profiles of various electrospun membrane morphologies were investigated. FIG. 10 shows the concept of a hybrid release system comprising a two-phase hybrid membrane where the nanofibers serve as a fast releasing medium and binder to hold microbeads while the embedded microbeads release drug in a much slow manner to achieve extended release duration.

Dual-syringe electrospinning. FIG. 1D illustrates an example of a dual syringe electrospinning (DSE) for composite and multilayer sandwiched membranes. The DSE setup comprises 2 syringes 103 and 121 loaded with different polymer solutions and drug concentrations (e.g., different PCL viscosities and DX concentrations). Multilayer and composite membranes with different morphologies can be obtained by alternating and simultaneous electrospinning approaches, respectively. DSE offers more degrees of freedom in process design parameters such as two polymers with different degradation time and concentrations, dual-drug release profiles, and sandwiched architectures with different morphology layers. Ultimately, a linear release can be realized by embedding drugs only in microbeads and using nanofibers as a diffusion barrier and packaging layer as shown in FIG. 1E. The triple-layered membrane shown can be further expanded with additional layers to realize different release profiles.

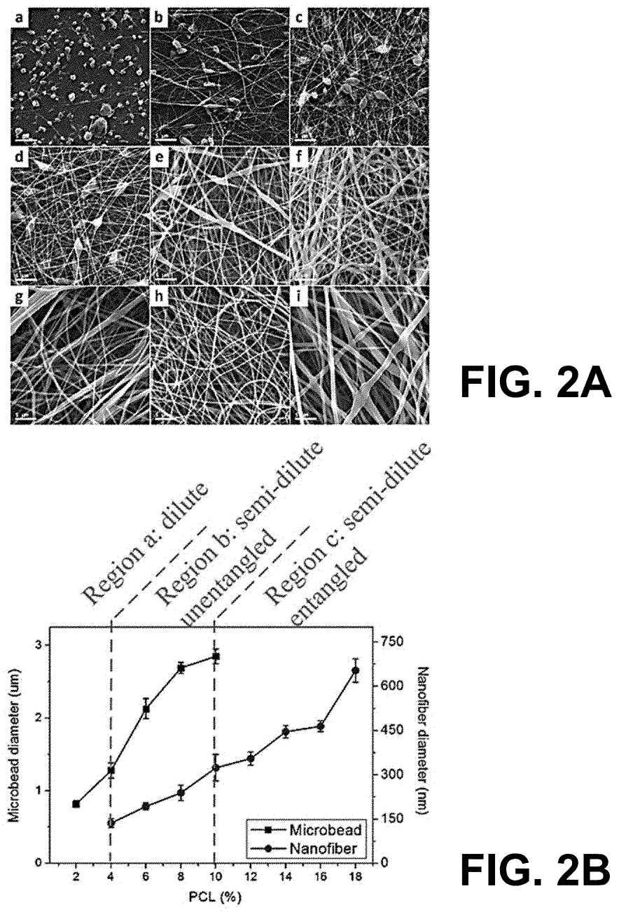

Single-phase membranes. Scanning electron microscope (SEM) images (a-i) of electrospun PCL microbeads/nanofibers with PCL concentrations varying from 2 w/v % to 18 w/v % are shown in FIG. 2A. All samples are electrospun at an electric field of 1 kV/cm and a TCD of 10 cm. The PCL concentrations were 2 w/v % (image a), 4 w/v % (image b), 6 w/v % (image c), 8 w/v % (image d), 10 w/v % (image e), 12 w/v % (image f), 14 w/v % (image g), 16 w/v % (image h), and 18 w/v % (image i). FIG. 2B shows the measured diameters of nanofibers and microbeads with the varying PCL concentrations and different morphologies. These are categorized in regions a, b, and c. Region a and c are identified as dilute and semi-dilute entangled solutions, respectively, which produce only single-phase membranes. In dilute region a, the microbead formation is due to low polymer concentration which results in the surface tension instability and low viscoelasticity. The average diameter of single-phase microbeads at 2 w/v % PCL was measured as 860 nm. Region c produces pure nanofibers with an average nanofiber diameter ranging from 310 nm to 670 nm as PCL concentration increases from 12 w/v % to 18 w/v %, respectively. Region b is the semi-dilute unentangled condition located between regions a and c, which produces a two-phase hybrid membrane comprising both microbeads and nanofibers. As the polymer concentration increases, the solution becomes more semi-dilute entangled which results in a decreasing microbead volume ratio and increasing microbead average diameter. Since the drug release rate is determined by the diffusion through the amorphous region of polymer matrix, a larger diameter microbead structure is preferred to extend the release profile.

Release measurements were conducted by soaking the DX loaded electrospun membranes in 5 ml of phosphate buffered saline (PBS) at room temperature. Dulbecco's phosphate buffered saline (PBS) was purchased from Mediatech, Inc. (Manassas, Va., USA). The drug release experiments were conducted by soaking 2.5 mg of DX loaded membrane in 5 ml of PBS at room temperature under sink conditions. The sink conditions were validated by ensuring that 100% of the loaded drug was released into the solution. The immersed membranes are placed on a shaker to keep the solution well-mixed (e.g., 2314 Lab rotator, Thermo Scientific, Waltham, Mass., USA). The concentrations of DX in the solution were determined by UV-Vis spectrophotometry in the detection range of 220-270 nm. The dynamic concentrations of DX in the release medium were determined periodically by measuring the absorbance optical spectra in the detection range of 220-270 nm wavelength using a UV-Vis spectrophotometer (Thermospectronic Genesys 10 UV, Rochester, N.Y., USA). The concentration of DX was determined by the least square fit between the measured and reference calibration spectra.

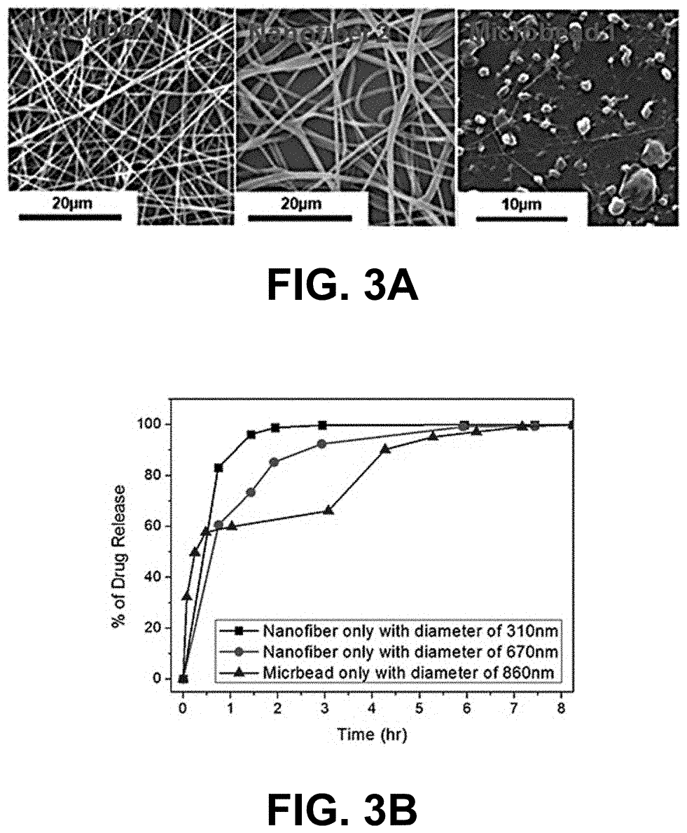

Three different PCL single-phase membranes (either nanofibers only or microbeads only) were prepared in this study with various solution conditions and electrospinning parameters. FIG. 3A shows SEM images of the single-phase membranes, which are identified as nanofiber 1, nanofiber 2, and microbead. Nanofiber 1 and microbead were electrospun by 16 w/v % and 2 w/v % of PCL dissolved in DCM/DMF with an electric field of 1 kV/cm, and a TCD of 12.5 cm. Nanofiber 2 was electrospun by 16 w/v % of PCL dissolved in acetone/ethanol with an electric field of 1 kV/cm, and a TCD of 12.5 cm. The average nanofiber diameters were measured as 310 nm and 670 nm for nanofiber 1 and 2, respectively. The larger nanofiber diameter obtained with nanofiber 2 is due to higher solvent evaporation rate of acetone/ethanol at room temperature compared to DCM/DMF solvent mixture in nanofiber 1. The solution jet of the nanofiber membrane 2 completely solidified before reaching the collector.

The DX release profiles of single-phase membranes of FIG. 3A with pure nanofibers or pure microbeads are shown in FIG. 3B. The nanofiber membrane 1 and 2 show a high initial burst rate, 80% release in one and two hours, respectively, which may be attributed to the small diameter of the nanofibers and the corresponding large surface to volume ratio. The microbead membrane took roughly 4 hours to reach 80% release. Almost all the drug was released within 8 hours.

The release durations of nanofiber 1 and 2 are approximately 2.5 hr and 6 hr, respectively. The short releasing durations may be attributed to the small diameters of nanofibers. The microbead with an average diameter of 860 nm has a release profile of 8 hr. Moreover, the microbead dispersion was observed after 3 hr, where the membrane was separated into smaller segments. Since the release mechanism is based on diffusion, the microbead release duration is decreased by dispersion when the contact area between microbeads and solution is enlarged. This indicates the release duration is affected by both the diameter of the structures and its aggregation morphology.

Two-phase hybrid membrane modeling. As drug release from drug embedded polymer relies on the diffusion process, a controlled release can be designed by accommodating the combination of differently sized drug embedded structures such as microbeads and nanofibers. A hybrid architecture comprising nanofibers and microbeads is observed when the PCL concentration is diluted to region b (FIG. 2B) resulting from reduced solution viscosity, which favors the formation of the microbeads due to the surface tension. The hybrid structure shows potential for longer release time due to the larger diameter of microbeads compared to nanofibers. Moreover, the ratio of the microbeads and nanofibers can be controlled by varying the applied voltage and TCD.

To further explore this, a series of experiments were conducted using 4 w/v % of PCL dissolved in 50:50 ratio of DCM/DMF with various applied voltages and TCD. A decrease in the volume fraction of microbeads with increasing TCD was observed. As TCD increases, the polymer solution experiences longer stretching time by electrostatic force, which results in decreasing the microbead diameter and higher surface to volume ratio of nanofiber.

A mathematical model can be developed for the prediction of a microbead diameter in the electrospun hybrid membrane. The model can be modified from one for a electrospun single-phase nanofiber membrane. The diameter of microbeads can be modeled by using the volume conservation and fluid-dynamic equations. The volume of the polymer can be expressed as the summation of the volume of the nanofiber and the volume of the microbead, which is given as:

4/3πr_{MB}³+πr_{NF}²L_{NF}=V, (1)

where r_{MB}, r_{NF}, L_{NF} and V are the microbead radius, nanofiber radius, nanofiber length, and polymer volume, respectively. The polymer solution flows through the needle with the assumption that the solution is incompressible Newtonian flow, two-dimensional fully developed laminar flow, constant circular cross-section, and has negligible surface roughness and gravity effects. The volumetric flow rate can be expressed as the following,

$$Q = \frac{V}{t} = {A}_{n} \frac{{L}_{n}}{t} = {A}_{n} {v}_{n} , \qquad ( 2 )$$

where Q, t, Lₙ, Aₙ, and νₙ are the flow rate, the solution travel time, the length of the needle, the cross-section area of the needle tip, and the velocity of the solution, respectively. The pressure gradient of solution can be derived by Hagen-Poiseuille equation as the following,

$$\frac{dp}{d L} = \frac{8 μ Q}{π {r}_{n}^{4}} , \qquad ( 3 )$$

where

$$\frac{dp}{d L} ,$$

μ and rₙ are the pressure gradient, the solution viscosity, and the needle radius, respectively.

The acceleration of the polymer jet can be derived using Coulomb's law and Newton's second law, as:

$$E = \frac{{F}_{1}}{q} = \frac{m a}{I t} , \qquad ( 4 )$$

where E, F₁, q, m, a, and I are the electric field, the electrostatic force, the electric charge, the mass of the polymer, the acceleration of polymer, and the current inside the polymer, respectively. Eq. (4) can be simplified as the following,

$$\begin{matrix}\frac{m}{t} = ρ \frac{V}{t} = ρ Q = ρ {A}_{n} {v}_{n} , & ( 5 ) \\ E = \frac{m a}{I t} = ρ {A}_{n} {v}_{n} \frac{a}{I} , & ( 6 )\end{matrix}$$

where ρ is the density of polymer jet. The final velocity of polymer jet at the collector can be described by Torricelli's equation as the following,

ν_{f}²=νᵢ²+2ad_{TCD}, (7)

where ν_{f}, νᵢ, and d_{TCD} are the final velocity of polymer jet, the initial velocity of polymer, which is the same as νₙ, and the TCD distance, respectively. The flow rate and total cross-section area of the polymer jets at the collector can be described by:

Q=A_{PCL}ν_{f}=πr_{PCL}²ν_{f}, (8)

where A_{PCL} and r_{PCL} are the effective PCL cross-section area and the radius right before it reaches the collector.

The electrostatic force (F₁) is balanced by surface tension force (F₂). The pressure gradient between the microbead and nanofiber can be derived from surface tension force as:

$$\frac{dp}{d L} = \frac{2 σ}{{r}_{M B} ( \frac{{L}_{N F}}{2} + {r}_{M B} )} , \qquad ( 9 )$$

where σ is the solution surface tension. Finally, Eq. (10) can be obtained by substituting Eq. (9) into Eq. (3). The values of μ and σ are obtained from the solution condition, and the values of ν_{f} and r_{PCL} are extracted from Eq. (7) and Eq. (8), respectively.

$$Q = π {r}_{P C L}^{2} {v}_{f} = \frac{π {r}_{P C L}^{4}}{8 μ} \frac{dp}{d L} = \frac{π {r}_{P C L}^{4}}{8 μ} \frac{2 σ}{{r}_{M B} ( \frac{{L}_{N F}}{2} + {r}_{M B} )} . \qquad ( 10 )$$

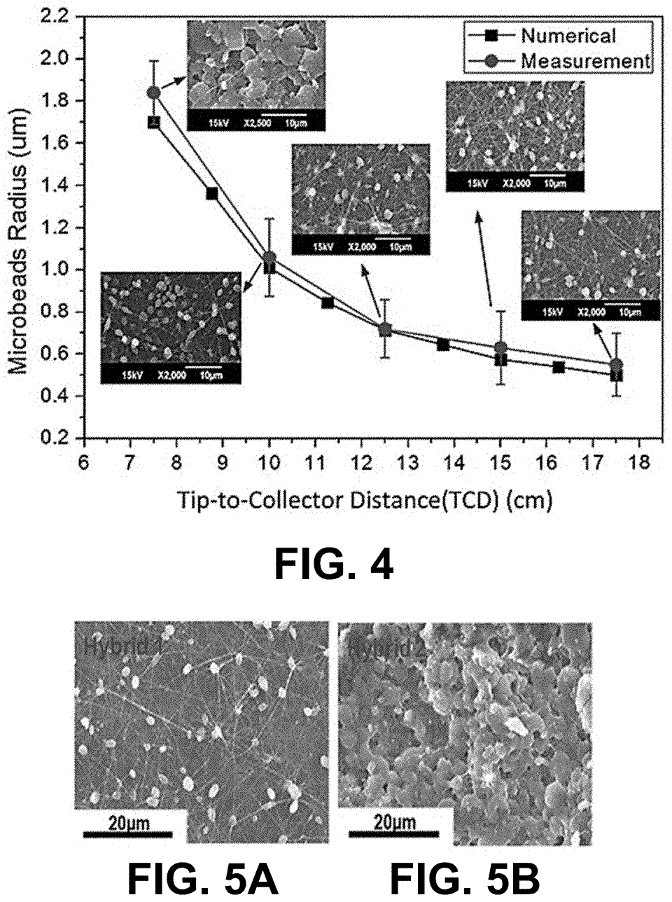

FIG. 4 shows the microbead diameter as a function of TCD from the experimental result and the analytical prediction. The experiments were conducted using 4 w/v % PCL solution with an electric field of 1 kV/cm. The derived analytical prediction shows good agreement with the experimental data measured by the software, ImageJ (National Institutes of Health, USA) with the assumptions of the same solution flow rate throughout the pumping and whipping process, and the constant electric field condition.

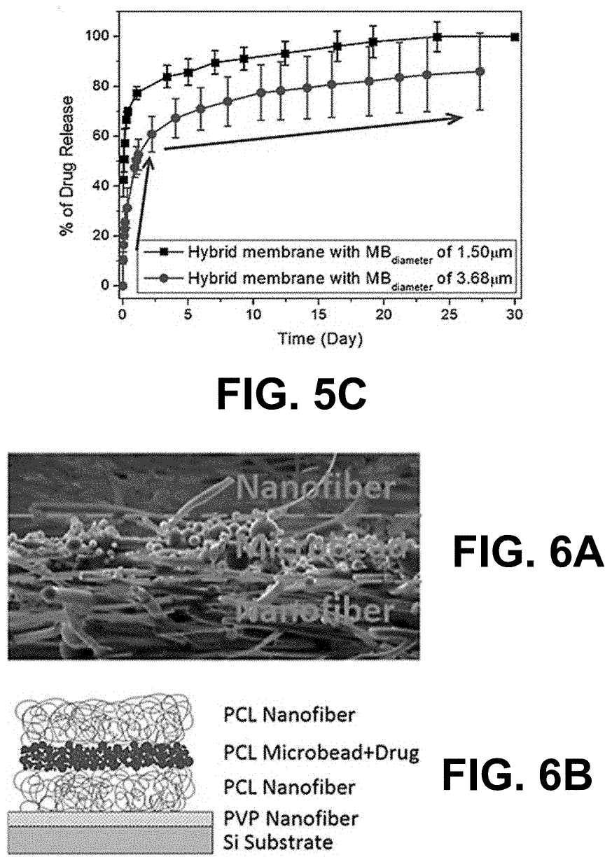

Two-phase hybrid membranes 1 and 2 were prepared with a PCL concentration of 4 w/v % in DCM/DMF, an electric field of 1 kV/cm, and a TCD of 12.5 cm and 7.5 cm, respectively. FIGS. 5A and 5B show SEM images of the two-phase hybrid membranes 1 and 2. The nanofiber and microbead diameters of hybrid membranes 1 and 2 are 108±51 nm and 1,560±600 μm, and 92±28 nm and 3,680±1,500 nm, respectively. The volume percentage of microbeads in hybrid membrane 1 and 2 are 40.15% and 91%, respectively.

FIG. 5C shows the DX release profiles of the two-phase hybrid membranes 1 and 2. The release durations of single-phase membranes, either nanofibers or microbeads, are less than 8 hours. The short releasing duration may be attributed to the small diameter of the nanofibers/microbeads. Both of the two-phase hybrid membranes show extended release profiles to about a month. Moreover, two-phase hybrid membrane 1 and 2 show a burst release with approximately 60% of drug released in first few hours and a much slower steady release after that. This is observed due to the hybrid structure comprising nanofibers and microbeads.

The microbeads with micrometer scale diameters show increased release time. The hybrid structure 1 with a microbead diameter of about 1.5 μm (FIG. 5A) shows approximately two days for 80% release while the remaining 20% is released over 30 days. On the other hand, the hybrid structure 2 with a microbead diameter of about 3.68 μm (FIG. 5B) shows almost 2.5 days for 60% release while the remaining release takes longer than a month. This shows a significant improvement with a prolonged release profile. However, it reports a still high initial burst rate (60%) in a short time frame compared with the entire release profile. Also, as the microbeads are loosely held by the small nanofibers, they are easily dispersed in the test media.

In the first stage of release, the burst may be attributed to the smaller diameters of nanofibers, and in the following stage, it may be attributed to the larger diameters of the microbeads. The release duration of both two-phase hybrid membranes is significantly prolonged as the diameters of microbeads are 5-10 times larger than those of the single-phase membranes. The extended release profile of over 1 month is obtained with a microbead diameter of about 3.68 μm. This two-phase hybrid system is suitable for applications that require a higher dosage level at an initial stage and a lower steady release thereafter.

Diffusivity and crystallinity study. With the experimental release profiles of single-phase and two-phase hybrid membranes, a mathematical release model was developed comprising microbeads and nanofibers. The model was modified from the diffusion monolithic systems for spherical and cylindrical structures. For single-phase membranes comprising only nanofibers or microbeads, the diffusivity equations of cylinders and spheres can be directly applied for nanofibers and microbeads as the following:

$$\frac{{M}_{t}}{{M}_{∞}} = 1 - \frac{32}{{π}^{2}} {\sum }_{n = 1}^{∞} \frac{1}{{q}_{n}^{2}} exp ( - \frac{{q}_{n}^{2}}{{r}_{N F}^{2}} Dt ) . \\ \frac{{M}_{t}}{{M}_{∞}} = 1 - \frac{6}{{π}^{2}} {\sum }_{n = 1}^{∞} \frac{1}{{n}^{2}} exp ( - \frac{D {n}^{2} {π}^{2} t}{{r}_{M B}^{2}} ) . \qquad ( 11 )$$

(12)
where Mₜ, M_{∞}, D, r_{MB}, r_{NF}, and qₙ are the cumulative drug release at time t, the total drug loaded, the drug effective diffusivity, the microbeads radius, the nanofibers radius, and the roots of the zero-order Bessel function of the first kind, respectively. However, to model the more complex two-phase hybrid system, which comprise both nanofibers and microbeads, additional parameters such as volume fractions of nanofiber (V_{NF}) and microbead (V_{MB}) are applied as the following:

$$\frac{{M}_{c}}{{M}_{∞}} = 1 - \{ {V}_{N F} \cdot [ {\sum }_{n = 1}^{∞} \frac{4}{{q}_{n}^{2}} exp ( - \frac{{q}_{n}^{2}}{{r}_{N F}^{2}} D t ) ] + {V}_{M B} \cdot [ \frac{6}{{π}^{2}} {\sum }_{n = 1}^{∞} \frac{1}{{n}^{2}} exp ( - \frac{D {n}^{2} {π}^{2} t}{{r}_{M B}^{2}} ) ] \} . \qquad ( 13 )$$

The resulting diffusivities of 725.5·10⁻¹¹ mm²/hr, 1,286.5·10⁻¹¹ mm²/hr, and 1,800.1·10⁻¹¹ mm²/hr were numerically extracted from the release profiles of single-phase nanofiber 1, nanofiber 2, and microbead membranes, respectively. In case of two-phase hybrid membranes, decreasing diffusivities of both nanofibers and microbeads were observed. For two-phase hybrid membrane 1 and 2, the diffusivities of nanofibers are 18.07·10⁻¹¹ mm²/hr and 2.59·10⁻¹¹ mm²/hr, and the diffusivities of microbeads are 14.27·10⁻¹¹ mm²/hr and 97.34·10⁻¹¹ mm²/hr, respectively. In comparison, a spin coated PCL thin film was prepared by spin coating the 16% (w/v) PCL in 1:1 mixture of DCM and DMF on a glass substrate and dried at room temperature. The extracted effective diffusivity of the spin coated thin film is 3.67×10⁻⁸ mm²/hr, which is roughly 2-3 orders larger than those of the electrospun membranes. The different diffusivities between conventional spin coated thin film and electrospun membranes may be attributed to polymer orientation and crystallinity.

Polymer crystallinity and orientation of the electrospun membrane can depend on several processing parameters, and it may affect the drug diffusivity. The crystallinity of electrospun nanofiber membranes may be higher compared to that of conventional spin coated thin films due to the high electrostatic stress during the whipping process. X-ray powder diffraction (XRD, PANalytical XPert Powder) was used and the degrees of crystallinity were analyzed by calculating the ratio of the area of the crystallite peak to the sum of the area of crystallite peak and amorphous. As a result, the crystallinity of spin coated thin film, nanofiber 1, nanofiber 2, hybrid 1, and hybrid 2 were 56%, 65%, 67%, 73%, and 75%, respectively.

The higher crystallinity in electrospun nanofiber/microbead membranes may be attributed to the shear stress generated during the electrospinning process that can align the polymer chains. Moreover, the two-phase hybrid membranes 1 and 2 show higher crystallinity compared to nanofiber membranes 1 and 2, which is consistent with the reduced diffusivities. Scherrer's equation was used to determine the crystallite size. The crystallite sizes of the spin coated thin film, nanofiber 1, nanofiber 2, hybrid 1, and hybrid 2 are 1.23 Å, 4.46 Å, 3.31 Å, 5.54 Å, and 5.91 Å, respectively. These results suggest that the electrospinning process can enhance both polymer crystallinity and crystallite sizes which results in reducing the effective diffusivity. In contrast to the conventional spin coated thin film, which shows a homogenous porous polymer, the electrospinning process is concluded to be a more suitable technique for fabricating a drug delivery carrier with a lower diffusivity and a longer release profile.

Effect of nanofiber barrier layer. The two-phase hybrid membranes show extended release profiles around a month with an approximate 60% of burst release at the beginning, which may cause toxicity with strong drug dosages. In order to minimize potential toxicity and enhance linearity of the release profile, a multilayer membrane architecture is proposed. The architecture comprises a drug loaded single-phase microbead layer which is sandwiched between PCL nanofiber barrier layers without drug loading. The proposed architecture can eliminate the fast release property of nanofibers while utilizing them as the barrier and binding layer to keep the microbead layer intact and bound during the drug release. The multilayer membrane was fabricated by DSE to study the effect of the nanofiber barrier layer. 18 w/v % PCL and 2 w/v % PCL with DX solutions were loaded in DSE for the alternating electrospinning process. Before PCL electrospinning, a 40 μm electrospun polyvinylpyrrolidone (PVP) nanofiber layer, which is water soluble was deposited on a silicon substrate as a sacrificial layer. The sandwiched PCL multilayer structure was easily released from the substrate when the PVP was dissolved in a PBS solution. After depositing a sacrificial PVP layer, a 40 μm thick PCL nanofiber layer was electrospun at 1 kV/cm with a TCD of 10 cm. Then, single-phase PCL microbeads with DX loaded were electrospun at 1 kV/cm and a TCD of 10 cm for a total of 2 mg of microbeads. Finally, another 40 μm thick PCL nanofiber layer was deposited, which serves as the top barrier layer with the same electrospinning condition.

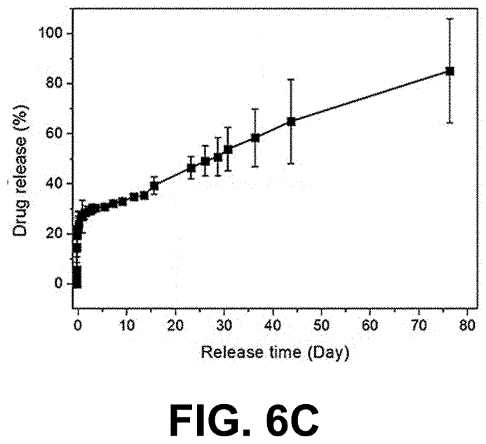

FIGS. 6A and 6B show an SEM image and a schematic of the fabricated multilayer membrane comprising nanofibers without drug and drug-loaded microbeads sandwiched between the nanofibers fabricated by DSE. The top and bottom PCL nanofiber layers have a thickness of approximately 40 μm and the middle PCL microbead layer has a thickness of approximately 30 μm.

FIG. 6C shows the release profile of the sandwiched multilayer membrane. The release profile shows a greatly reduced burst release and a highly linear steady release overall compared with both single-phase and two-phase membranes discussed in the previous sections. It shows a low initial burst of 30% for the first two days after which a linear release profile is obtained for 80 days up to 80% release. The extrapolated data shows approximately 120 days for 100% drug release. The reduced burst release is caused by the hydrophobicity of the PCL nanofiber barrier layer, which results in the delay of water penetration, and thus the diffusion of the drug through the barrier layer into the PBS is retarded.

After an initial period, drug was released in a much slower and more linear fashion, where the release rate is determined by the diffusion of the drug through the porosity of the nanofiber barrier layers. The linear steady release is ideal for a drug delivery system to provide an appropriate dosage throughout the entire release duration without potential over dosage, toxicity, and side effects. The DX release time has been greatly increased, e.g. extended up to 80 days by the additional nanofiber barrier layers. Moreover, the effective diffusivity of the sandwiched multilayer membrane was calculated to be 0.72·10⁻¹¹ mm²/hr which shows 2 and 3 orders of magnitude improvement compared to those of single-phase and two-phase hybrid membranes.

TABLE 1 shows the diameter, release duration, and effective diffusivity comparison of single-phase, two-phase hybrid, and sandwiched multilayer membranes. Single-phase membranes for both nanofibers and microbeads have rapid and short release profiles such as a few hours due to smaller diameters. Two-phase hybrid membranes show an extended release duration of 30 days. Two different release profiles are observed due to the different morphology and combination of nanofibers and microbeads, which shows the controllability of release profile based on tuning the electrospinning operating conditions. However, an approximate initial burst of 60% is observed due to the fast release of drug embedded in nanofibers. DSE technique was utilized to produce a sandwiched multilayer membrane where the drug is only loaded in the microbead layer to minimize the initial burst release and potential toxicity. The sandwiched multilayer membrane shows the longest release duration with a microbead diameter of only 0.86 μm. The PCL nanofiber barrier layers limit PBS solution intake and diffusion of DX to achieve an extended and linear release profile.

TABLE 1
Summary of nanofiber and microbead average diameters with corresponding
release time and effective diffusivity for all samples.

| | | | Release | | Diffusivity | Diffusivity |
|---|---|---|---|---|---|---|
| | NF diameter | MB diameter | time | | of nanofiber | of microbead |
| | [μm] | [μm] | (80%) | | (10⁻¹¹ mm²/hr) | (10⁻¹¹ mm²/hr) |
| Nanofiber 1 | 0.31 | N/A | 0.75 | hr | 725.5 | N/A |
| Nanofiber 2 | 0.67 | N/A | 1.92 | hr | 1,286.5 | N/A |
| Microbead | N/A | 0.86 | 3.70 | hr | N/A | 1,800.1 |
| Hybrid 1 | 0.11 | 1.52 | 3.40 | day | 18.07 | 14.27 |
| Hybrid 2 | 0.09 | 3.68 | 15.9 | day | 2.59 | 97.34 |
| Sandwiched | 0.61 | 0.86 | 46.7 | day | N/A | 0.72 |

The PCL based extended drug delivery systems with single phase, two phase, and sandwiched multilayer structures have been implemented using the SSE and DSE methods. PCL solutions with various concentrations and TCDs are studied to modulate the morphology of electrospun microbeads and nanofibers. The DX release profile and duration time can be designed and controlled by changing the morphology and architecture of the electrospun membrane. The two-phase hybrid membrane shows a two-phase release profile with a rapid release in the first few hours and a slow steady release for 30 days. This can be used for applications that require higher dosages upfront then a lower dosage for an extended time. The sandwiched multilayer membrane shows an extended release profile with a low burst release, a long release duration of 80 days, a highly linear release rate, and no microbead dispersion throughout 80 days. The two-phase hybrid and sandwiched multilayer membranes could be used as a treatment alternative to satisfy different application needs.

Fabrication of a triple-layered membrane comprising a dexamethasone (DX)-loaded polycaprolactone (PCL) microbead layer sandwiched between two PCL nanofiber layers has been demonstrated for highly extended linear drug release with low initial burst. Dual syringe sequential electrospinning can be used to produce the triple layers. A linear drug release of over 80 days and a significantly reduced initial burst of less than 30% is demonstrated. With the dual syringe electrospinning system, more degrees of freedom in the fabrication of a membrane with a customizable release profile can be realized.

Drug embedded microbead and nanofiber composite membranes for extended linear drug release can be fabricated using single and dual syringe electrospinning. First, single syringe electrospinning (SSE) with different polymer concentrations and tip-to-collector distances (TCD) is used to produce pure microbead, pure nanofiber, and hybrid microbead/nanofiber composite membranes. An analytical model for the prediction of the microbead diameter in the hybrid system is presented using the volume conservation and fluid dynamic equations. The calculated microbead diameters are compared with those of the experimented microbeads. A fabricated hybrid membrane with a drug release time of over one month is demonstrated. Second, dual syringe electrospinning (DSE) with more degrees of freedom in process parameters is explored to implement more diverse composite membranes. Independent selection of polymer types, viscosities, and drug loadings are exploited. A triple layer membrane comprising a drug loaded microbead layer sandwiched between two nanofiber layers with no drug loading is implemented by DSE, with which a further extended linear drug release of over 80 days and a significantly reduced initial burst release of less than 30% have been demonstrated.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A controlled release system, comprising:
a multilayer membrane comprising:
a first biocompatible nanofiber layer;
a microbead layer disposed on the first biocompatible nanofiber layer, the microbeads comprising a releasable agent; and
a second biocompatible nanofiber layer disposed over the microbead layer;
where the first and second biocompatible nanofiber layers support the microbead layer and provide a diffusion barrier configured to control a release profile of the releasable agent.

2. The system of claim 1, wherein the releasable agent is a drug.

3. The system of claim 1, wherein the first and second biocompatible nanofiber layers comprise electrospun nanofibers.

4. The system of claim 3, wherein the first biocompatible nanofiber layer comprises electrospun nanofibers having a first diameter and the second biocompatible nanofiber layer comprises electrospun nanofibers having a second diameter.

5. The system of claim 1, wherein the first and second biocompatible nanofiber layers comprise a biodegradable polymer.

6. The system of claim 5, wherein the biodegradable polymer is polycaprolactone (PCL).

7. The system of claim 1, wherein the microbead layer comprises microbeads in a volume percentage in a range from about 10% to about 99%.

8. The system of claim 7, wherein the volume percentage is in a range from about 25% to about 95%.

9. The system of claim 7, wherein the volume percentage is in a range from about 40% to about 91%.

10. The system of claim 1, wherein the release profile of the releasable agent comprises an initial burst and a linear release after the initial burst.

11. The system of 10, wherein the release rate of the initial burst is about 30% or less.

12. The system of 10, wherein the linear release extends over a period of about 80 days or more.

13. The system of claim 1, further comprising:
a second microbead layer disposed on the first biocompatible nanofiber layer; and
a third biocompatible nanofiber layer disposed over the second microbead layer.

14. The system of claim 13, wherein the second microbead layer comprises a second releasable agent.

15. The system of claim 1, wherein the microbead layer comprises a second releasable agent.
